(19) United States
(12) Patent Application Publication
(10) Pub. No.: US 2006/0226032 A1
(43) Pub. Date: Oct. 12, 2006

(54) Finger-mounted holder and strip-type blister package particularly useful therewith

(76) Inventor: Moshe Zalsman, Ramot-HaShavlm (IL)

Correspondence Address: Martin Moynihan;c/o ANTHONY CASTORINA, SUITE 207, 2001 JEFFERSON DAVIS HIGHWAY, ARLINGTON, VA, 22202, US

(21) Appl. No.: 11/102,653

(22) Filed: Apr. 11, 2005

(51) Int. Cl. A61B 19/02; A45C 11/00; A61C 19/00

(52) U.S. Cl. 206/63.5; 224/217; 206/37

(57) ABSTRACT

A holder for holding a substance to be applied by a user to a selected surface, includes a base member constructed for removably receiving a strip-type blister package having at least one compartment containing the substance to be applied by the user, and a finger-shaped mounting member joined to the base member for mounting the base member, and the strip-type blister package when received thereon, to a finger of the user to facilitate application of the substance of the blister package to the selected surface. Also described is a blister package containing a substance, particularly a dental substance, to be applied by a user to a selected surface, including a stiff base strip formed with at least a first compartment containing the substance to be applied to the selected surface, and with a second compartment containing a bonding agent first to be applied to the surface; and an overlying film bonded to the stiff base strip.

### FIELD AND BACKGROUND OF THE PRESENT INVENTION

The present invention relates to a finger-mounted holder for holding a substance to be applied by a user, and also to a strip-type blister package particularly useful with such a holder. The invention is especially useful for holders and blister packages containing dental substances, and is therefore described below with respect to such application, but it will be appreciated that the invention could also be used for holding and applying other substances, such as cosmetics, paint, etc.

It is known to use strip-type blister packages for holding various substances, including dental substances, in individual dosage forms. Examples of such blister packages for holding dental substances are described in U.S. Pat. No. 6,105,761, and the several patent documents identified in that patent. It is also known to use finger-mounted devices for conveniently holding various types of substances, including dental substances. Examples of the latter are described in U.S. Pat. Nos. 2,970,379, 3,327,391 and 4,844,308. However, the devices described in those patents are in the form of cups or trays, and are not suitable for strip-type blister packages.

### OBJECTS AND BRIEF SUMMARY OF THE PRESENT INVENTION

One object of the present invention is to provide a holder for a strip-type blister package containing a substance to be applied to a selected surface, which holder provides a number of advantages in facilitating the application of the substance by the user, as will be more particularly described below. Another object of the present invention is to provide a strip type blister package of a novel construction having advantages in a number of respects, as will also be more particularly described below.

According to one aspect of the present invention, there is provided a holder for holding a substance to be applied by a user to a selected surface, comprising: a base member constructed for removably receiving a strip-type blister package having at least one compartment containing the substance to be applied by the user, and a finger-shaped mounting member joined to the base member for mounting the base member, and the strip-type blister package when received thereon, to a finger of the user to facilitate application of the substance of the blister package to the selected surface.

According to further features in the preferred embodiment of the invention described below, the base member includes a supporting surface opposite to that facing the finger-shaped mounting member, formed with a peripheral inturned-flange, serving as peripheral frame for removably receiving the strip-type blister package. The peripheral frame is of rectangular configuration and is open at one side to permit the side insertion and removal of the blister package. The base member is of the same rectangular configuration as the peripheral frame and includes a central recessed surface for accommodating the at least one compartment of the strip-type blister package.

Such a holder holds the strip type blister package in a manner enabling the substance in the blister package to be conveniently applied by the user, and the used blister packages to be conveniently replaced. These features are particularly advantageous when the substances to be applied are dental substances, since such a holder frees the person's hand, normally used for holding the blister package, for other uses, e.g., for holding the patient's mouth in a particular position during the dental treatment. Similar advantages would also be present in other applications of the invention, e.g., for applying cosmetics, paint, or the like.

According to another aspect of the present invention, there is provided a blister package particularly useful with such a holder, the blister package comprising: a stiff base strip formed with at least a first compartment containing the substance to be applied to the selected surface, and with a second compartment containing a bonding agent to be applied to the selected surface before the substance is applied to promote the adhesion of the substance to the selected surface; and an overlying film bonded to the stiff base strip, the overlying film covering the compartments and the contents thereof.

According to further features in the described preferred embodiment, one end of the overlying film is peelable away from the base strip to expose the substance in the first compartment, and the opposite end includes a rupturable section overlying the second compartment to enable access of the bonding agent in the second compartment before the overlying film is peeled away to enable access to the substance in the first compartment.

According to still further features in the described preferred embodiment, the base strip is formed with a third compartment containing an additional quantity of the substance to be applied to the selected surface after application of the bonding agent. The third compartment is located between, and aligned with, the first and second compartments. The end of the overlying film adjacent to the first compartment includes a finger-tab to facilitate peeling away the overlying film from the first and third compartments to enable access to their contents. The opposite end of the overlying film includes a rupturable section overlying the second compartment to permit access to the bonding agent therein without peeling away the overlying film from the second compartment.

As indicated earlier, the invention is particularly useful for applying dental substances. In the preferred embodiment described below, the bonding agent and the substance are dental compositions.

Further features and advantages of the invention will be apparent from the description below.

### BRIEF DESCRIPTION OF THE DRAWINGS

The invention is herein described, by way of example only, with reference to the accompanying drawings, wherein:

FIG. 1 is an exploded three-dimensional view illustrating one form of finger-holder constructed in accordance with the present invention, a strip-type blister package to be received thereon, and an applicator to be used therewith;

FIG. 2 is a three-dimensional view illustrating the finger-holder of FIG. 1 with the strip-type blister package received thereon;

FIG. 3 is a sectional view illustrating the strip-type blister package of FIG. 1;

FIG. 4 illustrates the holder of FIGS. 1 and 2 on the user's finger, with the applicator rupturing the film overlying the bonding-agent compartment;

FIG. 5 illustrates the holder after the overlying film has been partially peeled away to expose one compartment containing the substance to be applied; and

FIG. 6 illustrates the holder after the overlying film has been further peeled away also to expose the additional compartment also containing the substance to be applied.

It is to be understood that the foregoing drawing and the description below, are provided primarily for purposes of facilitating understanding the conceptual aspects of the invention and possible embodiments thereof, including what is presently considered to be a preferred embodiment. In the interest of clarity and brevity, no attempt is made to provide more details than necessary to enable one skilled in the art, using routine skill and design, to understand and practice the described invention. It is to be further understood that the embodiments described are for purposes of example only, and that the invention is capable of being embodied in other forms and applications than described herein.

### DESCRIPTION OF A PREFERRED EMBODIMENT

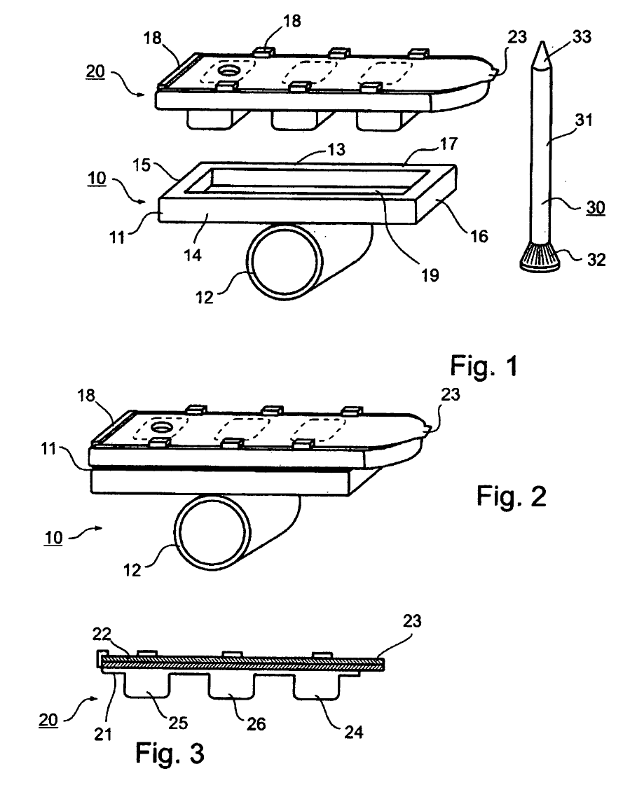

With reference first to FIG. 1, there is illustrated a holder, generally designated 10, for holding a substance to be applied by a user to a selected surface, and a strip-type blister package, generally designated 20, received on the holder and containing the substance to be applied. FIG. 1 also illustrates the applicator, generally designated 30, used with holder 10 and blister package 20 for applying the substance to the patient, as will be described more particularly below.

As indicated earlier, the invention is particularly, but not exclusively, useful by dentists or dental technicians for applying dental substances to a patient, and therefore blister package 20 contains unit-doses of several dental substances to be used for this purpose. Blister package 20 is removably received on holder 10 and contains sufficient dental substances for a single treatment, such that after the treatment, the blister package may be removed from the holder and replaced by another fresh blister package for treatment of another patient.

Holder 10 includes a base member 11 constructed for removably receiving the strip-type blister package 20 containing the substance to be applied by the user. Holder 10 further includes a finger-shaped mounting member 12 joined to base member 11 for mounting the base member, and the blister package 20 when received thereon, to the finger of a user to facilitate application of the substance of the blister package.

Base member 11 is of rectangular configuration. It includes a pair of opposed longitudinal sides 13, 14, and a pair of opposed transverse sides 15, 16, of shorter length than the longitudinal sides. The upper surface 17 of base member 11, i.e. the surface facing away from the finger-shaped mounting member 12, is formed with an in-turned flange 18 along its two longitudinal sides 13, 14, and along one transverse side 15, to define a peripheral frame which is open at side 16 to permit insertion and removal of the strip-type blister package from side 16 of the base member. Preferably the in-turned flange 18 along the two longitudinal sides of the base member are interrupted, whereas the flange along the transverse side 15 of the base member is continuous, so as to facilitate the edge insertion of the strip-type package 20 from the open transverse side 16 of the base member.

It will thus be seen that when the strip-type blister package 20 is applied to base member 11 via the open end 16 of the base member, the blister package will be securely held in place between the upper surface 17 of base member 11 and the intoned flanges 18 along the three sides 13, 14 and 15 of the base member. Preferably, the central region of surface 17 of base member 11 is recessed, as shown at 19, in order to accommodate the compartments in the strip-type blister package when received on the base member. Recess 19 may be a single large recess to accommodate all the compartments, or may be separate recesses, one for each of the compartments in the blister package.

The blister package 20 includes a stiff base strip 21, and an overlying film 22 bonded to the base strip. One end of the overlying film 22 is formed with a finger-tab 23 to facilitate peeling the film away from the base strip, as will be described more particularly below.

Base strip 21 is of a stiff plastic material and is formed with three cavities or compartments 24, 25, 26 for containing unit-doses of the various substances included within the blister package. In the embodiment illustrated, compartment 24, at the end of the base strip adjacent to the finger-tab 23, is filled with the dental substance to be applied to the selected surface, and compartment 25 at the opposite end of the base strip is filled with a bonding agent to be applied to the selected surface, before the substance of compartment 24 in order to promote the adherence of the latter substance to the selected surface. The middle compartment 26 is also to contain the same dental substance as compartment 24 so as to permit the application of such substance in the form of distinct layers, if desired.

The portion of film 22 overlying compartment 26, to contain the bonding agent, is provided with a rupturable section 27, which may be ruptured before the overlying film 22 is peeled away from the base strip 21. Thus, as will be described more particularly below, section 27 may be first ruptured, e.g., by use of the applicator 30, to permit small quantities of the bonding agent to be first applied to the selected surface to be treated; and then the overlying film 22 may be peeled away to expose the dental substance within compartments 24 and 26 for application to the selected surface to be treated.

As shown in FIG. 1, applicator 30 may be in the form of a brush, spatula, or the like. It includes a handle 31 having a brush head or spatula 32 at one end. The opposite end of handle 31 is somewhat pointed, as shown at 33, to enable that end to be used for rupturing film section 27 overlying compartment 25 containing the bonding agent.

The manner of using holder and blister package will be apparent from the above description. Thus, the strip type blister package 20 contains, in one end compartment 24 and in the middle compartment 26, the dental substance to be applied to the patient. The blister package also contains a bonding agent in the opposite end compartment 25. Blister package 20 is applied to base member 11 of holder 10 via the open end 16, (i.e., the transverse edge of the base member lacking an in-turned flange) such that the blister package is firmly retained between the upper surface 17 and the inwardly-facing flanges 18 of the base member.

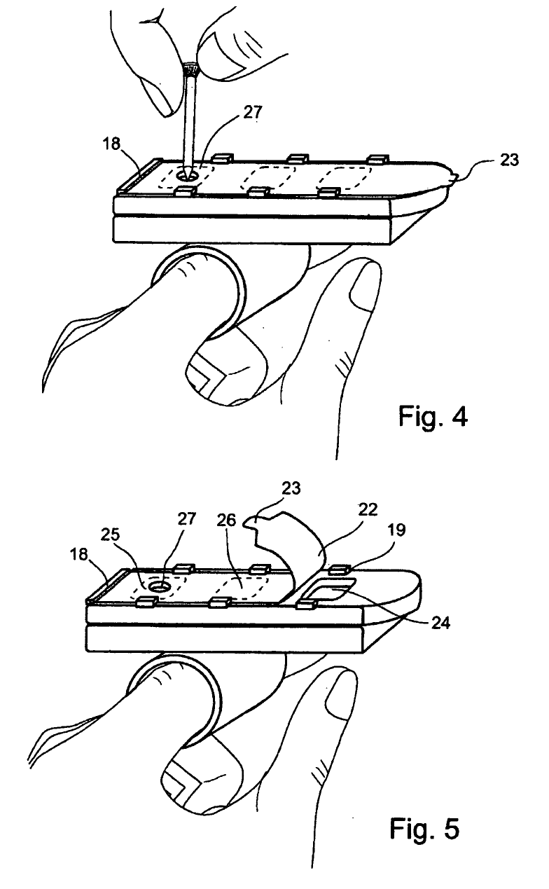

The pointed end 33 of applicator 30 may then be used to rupture section 27 of film 22 overlying compartment 25 containing the bonding agent as shown in FIG. 4. This may be done before or after the holder is applied to the user's finger.

With the holder applied to the user's finger, and with portion 27 of film 22 ruptured, the user may then dip the brush or spatula end 32 of applicator 30 into the bonding agent within compartment 25 and apply some of the bonding agent to the surface to be treated.

Finger-tab 23 may be then be grasped and pulled outwardly to peel-away the overlying film 22 only from compartment 24, to expose the dental substance within that compartment as shown in FIG. 5. Preferably another applicator may then be dipped into the dental substance within compartment 24 and applied to the surface to be treated.

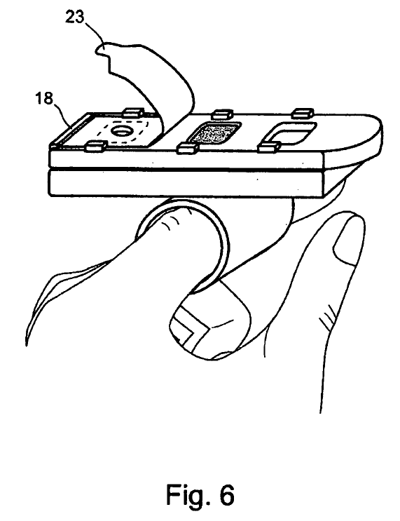

If more substance is required, finger-tab 23 may be pulled further away from the base strip 21 to uncover compartment 26, and thereby to enable the additional dental substance within that compartment to be similarly applied as shown in FIG. 6.

The dental substance within compartments 24 and 26, as well as the bonding agent within compartment 25, are preferably an appropriate light-activated (e.g., ultraviolet-activated) dental composition. The treatment site may be exposed to the ultraviolet light after each application of the substances agent within compartments 25, 24 and 26; or the treatment site may be exposed to ultraviolet light after all the foregoing dental compositions have been applied to the treatment site.

As one example, the bonding agent within compartment 25 may be a dental/enamel bonding agent called “PRIMA 2000”, supplied by B.J.M. Laboratories, Ltd. of Or Yehuda, Israel; and the dental substance with compartments 24 and 26 may be a restoration component called “Pro Fil”, supplied by the same company.

While the invention has been described with respect to one preferred embodiment, it will be appreciated this is set forth merely for purposes of example, and that many variations and other applications of the invention may be made. For example, the described holder and blister package can be used for applying other types of dental compositions. In addition, they may be used applying other materials to selected surfaces, for example cosmetics.

Many other variations, modifications and applications will be apparent.

What is claimed is:

1. A holder for holding a substance to be applied by a user to a selected surface, comprising:
a base member constructed for removably receiving a strip-type blister package having at least one compartment containing the substance to be applied by the user;
and a finger-shaped mounting member joined to said base member for mounting the base member, and the strip-type blister package when received thereon, to a finger of the user to facilitate application of the substance of the blister package to said selected surface.

2. The holder, according to claim 1, when said base member includes a supporting surface, opposite to that facing the finger-shaped mounting member, formed with a peripheral inturned-flange, serving as peripheral frame for removably receiving said strip-type blister package, said peripheral frame being open at a side thereof to permit the side insertion and removal of said blister package.

3. The holder, according to claim 2, wherein said peripheral frame is of rectangular configuration and is open at said side to permit the side insertion and removal of said blister package.

4. The holder according to claim 3, wherein said peripheral frame is of rectangular configuration and includes a pair of opposed longitudinal sides, and a pair of opposed transverse sides of shorter length than said longitudinal sides, one of said transverse sides being the open side to permit the side insertion and removal of said strip type blister package.

5. The holder according to claim 4, wherein said base member is of the same rectangular configuration as said peripheral frame and includes a central recessed region for accommodating the at least one compartment of the stripe-type blister package.

6. The holder according to claim 1, in combination with a strip-type blister package removably received on said base member.

7. The holder according to claim 6, wherein said strip-type blister package includes:
a stiff base strip formed with at least first and second compartments containing substances to be applied by the user to the selected surface;
and an overlying film bonded to said stiff base strip to cover said compartments and the substances therein.

8. The holder according to claim 7, wherein said overlying film is peelable away from said base strip to expose the substance in said first compartment, and includes a rupturable section overlying said second compartment to expose the substance therein without peeling away said overlying film.

9. The holder according to claim 8, wherein the end of said overlying film adjacent to said first compartment includes a finger-tab to facilitate peeling said overlying film away from said first compartment without peeling said film away from said second compartment.

10. The holder according to claim 7, wherein the substance of said second compartment is a bonding agent to be applied to the selected surface before the substance of said first compartment in order to promote the adherence of the substance of said first compartment to said selected surface.

11. The holder according to claim 10, wherein said stiff base strip is formed with a third compartment containing an additional quantity of the substance of the first compartment, said third compartment being located between, and aligned with, said first and second compartments.

12. The holder according to claim 11, wherein one end of said overlying film, adjacent to said first compartment, includes a finger-tab to facilitate peeling away that end of the overlying film from said first and third compartments to enable access to their contents; the opposite end of said overlying film including a rupturable section overlying said second compartment to permit access to the bonding agent therein without peeling away that end of the overlying film from said second compartment containing said bonding agent.

13. The holder according to claim 10, wherein said bonding agent and said substance are dental compositions.

14. A blister package containing a substance to be applied by a user to a selected surface, comprising:
a stiff base strip formed with at least a first compartment containing the substance to be applied to the selected surface, and with a second compartment containing a bonding agent to be applied to the selected surface before said substance is applied to promote the adhesion of said substance to the selected surface;
and an overlying film bonded to said stiff base strip, said overlying film covering said compartments and the contents thereof.

15. The blister package according to claim 14, wherein one end of said overlying film is peelable away from said base strip to expose the substance in said first compartment, and the opposite end includes a rupturable section overlying said second compartment to enable access to the bonding agent in said second compartment before said overlying film is peeled away to enable access to the substance in said first compartment.

16. The blister package according to claim 15, wherein said one end of the overlying film adjacent to said first compartment, containing the substance to be applied by the user to the selected surface, includes a finger-tab to facilitate peeling said end of the overlying film away from said end of the base strip, and thereby to enable access to the contents of said first compartment without peeling the overlying film away from said second compartment containing said bonding agent.

17. The blister package according to claim 14, wherein said stiff base strip is formed with a third compartment containing an additional quantity of the substance to be applied to the selected surface after application of said bonding agent.

18. The blister package according to claim 17, wherein said third compartment is located between, and aligned with, said first and second compartments.

19. The blister package according to claim 18, wherein said one end of the overlying film adjacent to said first compartment includes a finger-tab to facilitate peeling away said one end of the overlying film from said first and third compartments to enable access to their contents; the opposite end of said overlying film including a rupturable section overlying said second compartment to permit access to the bonding agent therein without peeling away said opposite end of the overlying film from said second compartment containing said bonding agent.

20. The blister package according to claim 14, wherein said bonding agent and said substance are dental compositions.
